(19) United States
(12) Patent Application Publication
(10) Pub. No.: US 2023/0058297 A1
(43) Pub. Date: Feb. 23, 2023

(54) FABRICATION OF 3D-PRINTED FRACTURE-SPECIFIC ORTHOPAEDIC CAST

(71) Applicant: 3D IMPLANTS, INCUBATEE, FOUNDATION FOR INNOVATION & RESEARCH AT SASTRA TECHNOLOGY BUSINESS INCUBATOR, Thanjavur (IN)

(72) Inventors: Swaminathan SETHURAMAN, Thanjavur (IN); Anuradha SUBRAMANIAN, Thanjavur (IN); Vishal KANNAN, Thanjavur (IN)

(73) Assignee: 3D IMPLANTS, INCUBATEE, FOUNDATION FOR INNOVATION & RESEARCH AT SASTRA TECHNOLOGY BUSINESS INCUBATOR, Thanjavur (IN)

(21) Appl. No.: 17/797,985

(22) PCT Filed: Feb. 5, 2021
(86) PCT No.: PCT/IN2021/050119

§ 371 Date: Aug. 5, 2022

(30) Foreign Application Priority Data
Feb. 5, 2020 (IN) 202041005024

(51) Int. Cl. A61F 5/058; A61N 5/06; G06F 30/10; B33Y 50/00; B33Y 80/00; B29C 64/386

(52) U.S. Cl. CPC A61F 5/05866; A61N 5/0616; G06F 30/10; B33Y 50/00; B33Y 80/00; B29C 64/386; A61H 1/00

(57) ABSTRACT

A method (1100) of fabricating a personalised orthopaedic cast (900) is disclosed. The method (1100) includes 3D scanning of a body part of a user, generating a Computer Aided Design (CAD) of an orthopaedic cast (900) for the scanned body part, and simulating real-life conditions to determine mechanical stability of the modelled cast. The mechanical stability is determined through Finite Element Analysis (FEA). The method (1100) includes determining whether the mechanical stability of the modelled cast is acceptable. The method (1100) includes finalising the CAD model when the mechanical stability of the modelled cast is found to be acceptable. The method (1100) includes 3D printing the finalised CAD model to fabricate the personalised orthopaedic cast (900).

### FIELD OF THE INVENTION

The present disclosure relates to orthopaedic casts and more particularly, relates to fabrication of a 3D-printed fracture-specific orthopaedic cast.

### BACKGROUND

Bone fracture is a common injury across the globe that affects more than 8.9 million people annually leading to one fracture every three seconds. Arm fractures are reported to have highest incidences accounting for half of all bone fractures. The symptoms include uncontrolled swelling, pain, deformity that requires immobilization and adequate stabilization to fix the bones precisely till the healing completes. Orthopaedic devices such as splints and casts are mainly used along with the cotton and padding materials to support the fractured bones in a proper alignment for 4 to 8 weeks. However, these conventional cast systems such as plasters and fiberglass provoke cast-associated discomforts such as numbness, pressure sores, swelling, tingling, and itching due to its overweight (700-1200 g), non-washability and poor breathability. Further non-porous nature of the traditional casts prevents the physicians to monitor the healing process of any cutaneous wounds formed either by casting or by trauma-induced fracture.

Particularly, fracture healing involves six stages starting from acute inflammation, recruitment of mesenchymal cells, generation of callus, revascularization and neoangiogenesis, mineralization and resorption of cartilaginous bone and finally bone remodeling (Marsell et al., 2011). The entire healing process takes a minimum of 6-8 weeks with bone remodeling phase extending based on the individual's health and lifestyle. The general course of treatment includes immobilizing the fractured bones by application of casts and splints for the entire healing phase of 6-8 weeks (Meena et al., 2014). The application of traditional casts made of Plaster of Paris (POP) for 6-8 weeks provoke a number of cutaneous complications including skin maceration, blisters, pressure sores, compartment syndrome, deep vein thrombosis and other secondary infections (B. Szostakowski et al., 2017) due to its heaviness (750-1000 g) and lack of breathability. Moreover, POP requires skilled professionals for its application. It has a setting stage and a hardening stage, and any procedural mistake will lead to the loss of mechanical stability (Boyd et al., 2009; Parmar et al., 2014) and improper immobilization of the underlying hand leading to a malunion.

Although fiber-glass casts become an alternate to POP due to its lesser weight (500-700 g), poor ventilation, sharp edges with the requirement of special cast saw for the removal of cast and any procedural mistake again will induce damage to the underlying hand (Inglis et al., 2013). Further, the applied casts tend to get loosened in the due course of healing due to gradual reduction of swelling demands the periodic removal followed by the application of new casts till the healing completes.

Recent advances such as synthetic casts are being developed to overcome the complications of traditional casts (Bullen et al., 2017; Zhu et al., 2019). Though many are still in their research stage, some of them hit the commercial market. One such is FlexiOH™, developed by an Indian based startup, which is a silicone-based cast. The application of FlexiOH is a two-step procedure that gets hardened only upon irradiation with a specific wavelength of electromagnetic radiation (Company and Data, 2017). However, the extent of curing determines the mechanical stability of the cast and improper curing at some places produce variation in the mechanical strength across the cast leading to improper immobilization of the fractured hand. Moreover, these are available only in three standard sizes and lack individual personalization. Though it has better properties than POP and fiberglass such as reduced weight (minimum 350 g) and decreased chance of developing pressure sores, it was still quite heavy and had improper ventilation due to the presence of cotton padding.

Another study tried out a hybrid model that had two parts—a 3D printed inner frame and an outer covering built using injection molding (Kim and Jeong et al., 2015). Though this hybrid strategy reduces the fabrication cost, improper ventilation with the lack of fracture-specific personalization are the major limitations. Also, the use of injection molding encumbers one to create personalized and complex designs with high accuracy and precision. Honeycomb patterns in the cast developed by modified K-mean facet clustering algorithm followed by 3D printing increases ventilation as well as the strength to weight ratio, but, failed to analyze the mechanical stability of the design (Ahsan and Khoda et al., 2018). Hence both traditional as well as synthetic cast lacks fracture precision, breathable, washable, and readily wearable casting solutions for immobilization of bones.

### SUMMARY

This summary is provided to introduce a selection of concepts, in a simplified format, that are further described in the detailed description of the invention. This summary is neither intended to identify key or essential inventive concepts of the invention and nor is it intended for determining the scope of the invention.

In an embodiment of the present disclosure, a method of fabricating a personalised orthopaedic cast is disclosed. The method includes 3D scanning of a body part of a user which is to be immobilized, generating a Computer Aided Design (CAD) of an orthopaedic cast for the scanned body part, and simulating real-life conditions to determine mechanical stability of the modelled cast. The mechanical stability is determined through Finite Element Analysis (FEA). The method includes determining whether the mechanical stability of the modelled cast is acceptable. The method includes finalising the CAD model when the mechanical stability of the modelled cast is found to be acceptable. The method includes 3D printing of the finalised CAD model to fabricate the personalised orthopaedic cast.

In another embodiment of the present disclosure, a 3D-printed orthopaedic cast for a body part of a user is disclosed. The orthopaedic cast includes a base component formed of a first part and a second part adapted to be connected with the first part such that the base component is wrapped around the body part. The base component includes a predefined grid pattern forming pores on a surface. The orthopaedic cast includes a Velcro-based lock adapted to connect the first part with the second part to form the base component. The 3D-printed orthopaedic cast is formed after generation of a Computer-Aided Design (CAD) and Finite Element Analysis (FEA) of the modelled cast.

In the present disclosure, a novel approach for fabricating a personalized orthopaedic cast for fracture specific application is disclosed. Simulation tools and 3D printing technology are used to form the personalised orthopaedic cast. The objective of the present invention is to outperform the conventional casts by introducing properties such as breathability, customizability, mechanical stability, low weight, and aesthetically pleasing design without compromising the immobilization of the fractured hand. A sequential methodology from scanning the individual's arm to develop a CAD model of the cast and simulating it using Finite Element Analysis (FEA) before finally printing it using FDM approach is disclosed. The scanning of individual's arm seeded the way to create a customized CAD model. The strategy was focused more on design-based approach and its importance in deciding the mechanical firmness of the cast. The simulation tool enabled us to simulate various real time conditions to check the mechanical stability of the designed cast more feasibly, overcoming the trial-and-error approach. The function of the cast attributes to its ability to immobilize the fractured bone by limiting the stress transfer and enhancing the stress distribution. However, introduction of the pores in the design to enhance the ventilation may compromise the mechanical strength of the case especially on the fractured region, which would completely be avoided by testing the mechanical robustness and the ability of the design to distribute the stress uniformly by finite element analysis prior to 3D printing. The total deformation and equivalent stress distribution of the cast quantitates its stress transfer and stress distribution capability, respectively.

Additive manufacturing helped in building highly complex structures with high accuracy and precision, and in realizing the mechanically approved design in real time. It also facilitates to create and integrate various lock models to give proper fitting and enable adjustability based on the rate of swelling of the underlying hand which could completely avoid the frequent casting procedures. Further, the design enables us to integrate the specific probes to deliver the Infra-red light/vibration therapy at fracture-specific region. We have initially created a Velcro-based model and integrated with the mechanically approved design to enable user friendly application procedure. Thus, we have created a methodology for optimizing mechanically robust CAD modelled design from FEA and synchronizing it with the printing parameters to create a common pipeline in the whole procedure of fabricating a fracture-specific orthopaedic cast. This strategy gave us the flexibility to design a cast personalized to the patient and to the fracture as well.

To further clarify advantages and features of the present invention, a more particular description of the invention will be rendered by reference to specific embodiments thereof, which is illustrated in the appended drawings. It is appreciated that these drawings depict only typical embodiments of the invention and are therefore not to be considered limiting of its scope. The invention will be described and explained with additional specificity and detail with the accompanying drawings.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 illustrates a schematic representation of the experimental strategy, according to an embodiment of the present disclosure;

FIG. 2 illustrates a processed scanned image of the patient's arm with region of interest (ROI), according to an embodiment of the present disclosure;

FIG. 3 illustrates CAD modelled cast designs, according to an embodiment of the present disclosure;

FIG. 4 illustrates Finite Element Analysis of total deformation, according to an embodiment of the present disclosure;

FIG. 5 illustrates a comparative analysis of total deformation with design and material variability, according to an embodiment of the present disclosure;

FIGS. 6A and 6B illustrate Finite Element Analysis of stress distribution of various designs, according to an embodiment of the present disclosure;

FIGS. 7A and 7B illustrate Finite Element Analysis of stress distribution of various designs, according to an embodiment of the present disclosure;

FIG. 8 illustrates comparative analysis of average stress distribution with design and material variability, according to an embodiment of the present disclosure;

FIG. 9 illustrates an optimized mechanically robust design with Velcro based lock, a 3D printed Cast, and Personalized hand-cast, according to an embodiment of the present disclosure;

FIG. 10 illustrates fracture-specific breathable and washable orthopaedic cast integrated with a probe adapter for adjuvant photo/vibration therapy, according to an embodiment of the present disclosure; and

FIG. 11 illustrates a flow chart depicting a method of fabrication of the personalised orthopaedic cast, according to an embodiment of the present disclosure.

Further, skilled artisans will appreciate that elements in the drawings are illustrated for simplicity and may not have been necessarily been drawn to scale. For example, the flow charts illustrate the method in terms of the most prominent steps involved to help to improve understanding of aspects of the present invention. Furthermore, in terms of the construction of the device, one or more components of the device may have been represented in the drawings by conventional symbols, and the drawings may show only those specific details that are pertinent to understanding the embodiments of the present invention so as not to obscure the drawings with details that will be readily apparent to those of ordinary skill in the art having benefit of the description herein.

### DETAILED DESCRIPTION OF FIGURES

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates. Unless otherwise defined, all technical and scientific terms used herein have the same meaning as commonly understood by one of ordinary skilled in the art to which this invention belongs. The system, methods, and examples provided herein are illustrative only and not intended to be limiting.

For example, the term “some” as used herein may be understood as “none” or “one” or “more than one” or “all.” Therefore, the terms “none,” “one,” “more than one,” “more than one, but not all” or “all” would fall under the definition of “some.” It should be appreciated by a person skilled in the art that the terminology and structure employed herein is for describing, teaching, and illuminating some embodiments and their specific features and elements and therefore, should not be construed to limit, restrict, or reduce the spirit and scope of the present disclosure in any way.

For example, any terms used herein such as, “includes,” “comprises,” “has,” “consists,” and similar grammatical variants do not specify an exact limitation or restriction, and certainly do not exclude the possible addition of one or more features or elements, unless otherwise stated. Further, such terms must not be taken to exclude the possible removal of one or more of the listed features and elements, unless otherwise stated, for example, by using the limiting language including, but not limited to, “must comprise” or “needs to include.”

Whether or not a certain feature or element was limited to being used only once, it may still be referred to as “one or more features” or “one or more elements” or “at least one feature” or “at least one element.” Furthermore, the use of the terms “one or more” or “at least one” feature or element do not preclude there being none of that feature or element, unless otherwise specified by limiting language including, but not limited to, “there needs to be one or more . . . ” or “one or more element is required.”

Unless otherwise defined, all terms and especially any technical and/or scientific terms, used herein may be taken to have the same meaning as commonly understood by a person ordinarily skilled in the art.

Reference is made herein to some “embodiments.” It should be understood that an embodiment is an example of a possible implementation of any features and/or elements of the present disclosure. Some embodiments have been described for the purpose of explaining one or more of the potential ways in which the specific features and/or elements of the proposed disclosure fulfil the requirements of uniqueness, utility, and non-obviousness.

Use of the phrases and/or terms including, but not limited to, “a first embodiment,” “a further embodiment,” “an alternate embodiment,” “one embodiment,” “an embodiment,” “multiple embodiments,” “some embodiments,” “other embodiments,” “further embodiment”, “furthermore embodiment”, “additional embodiment” or other variants thereof do not necessarily refer to the same embodiments. Unless otherwise specified, one or more particular features and/or elements described in connection with one or more embodiments may be found in one embodiment, or may be found in more than one embodiment, or may be found in all embodiments, or may be found in no embodiments. Although one or more features and/or elements may be described herein in the context of only a single embodiment, or in the context of more than one embodiment, or in the context of all embodiments, the features and/or elements may instead be provided separately or in any appropriate combination or not at all. Conversely, any features and/or elements described in the context of separate embodiments may alternatively be realized as existing together in the context of a single embodiment.

Any particular and all details set forth herein are used in the context of some embodiments and therefore should not necessarily be taken as limiting factors to the proposed disclosure.

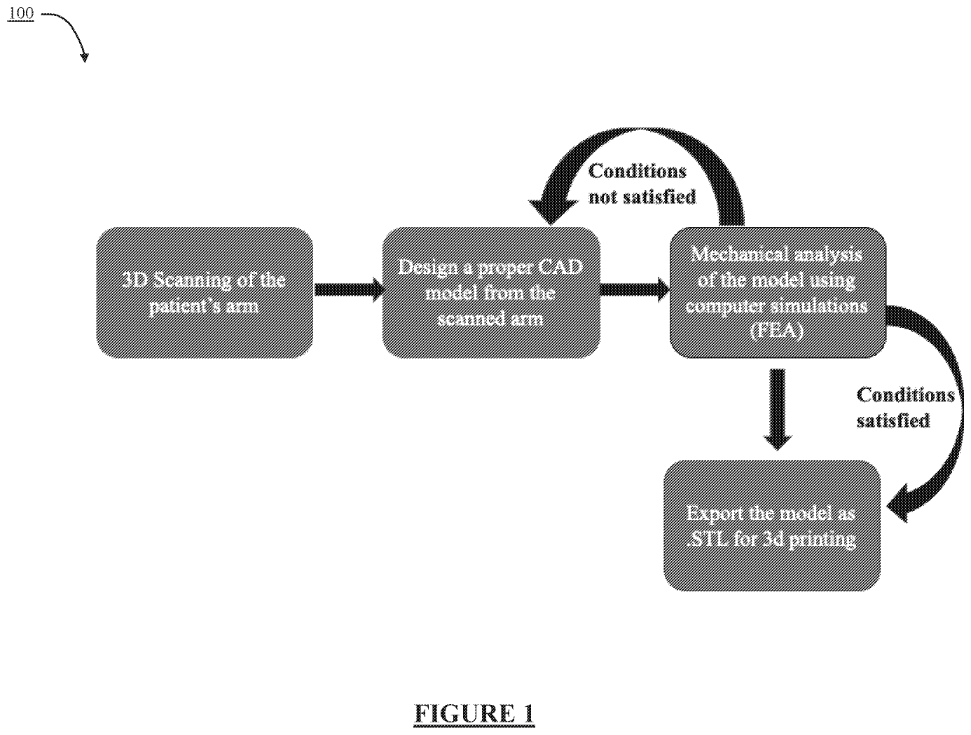

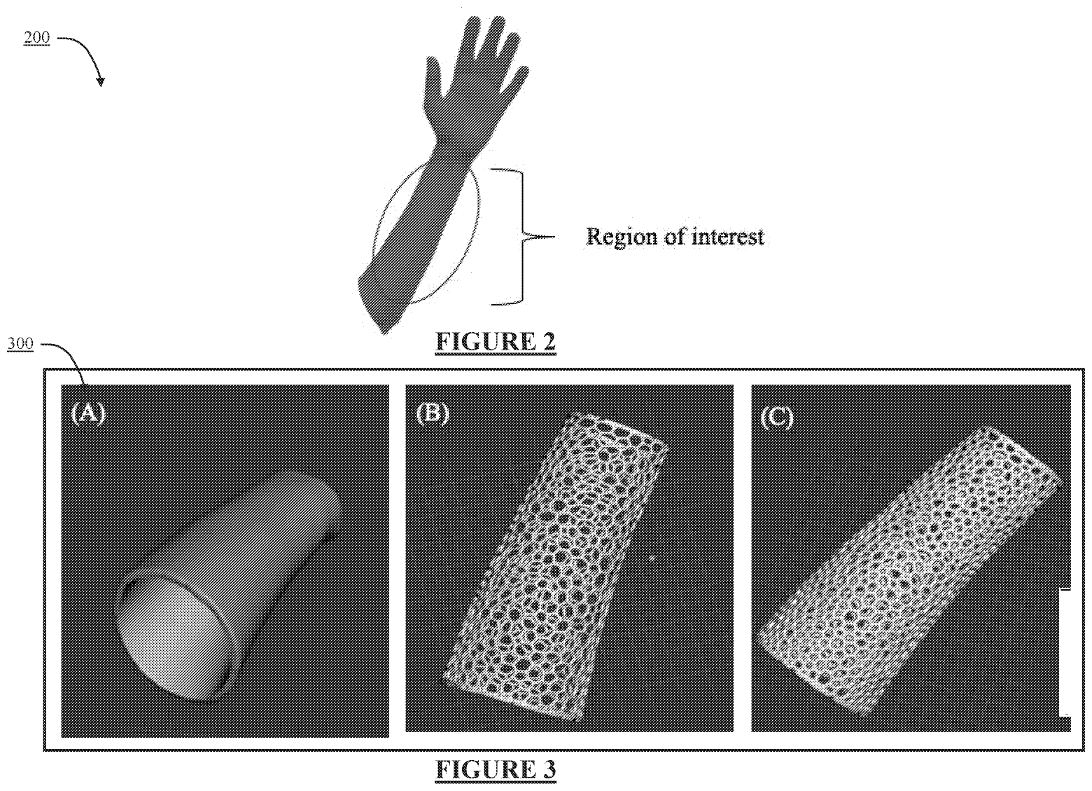

For the sake of clarity, the first digit of a reference numeral of each component of the present disclosure is indicative of the Figure number, in which the corresponding component is shown. For example, reference numerals starting with digit “1” are shown at least in FIG. 1. Similarly, reference numerals starting with digit “2” are shown at least in FIG. 2.

The present disclosure involves simulation tools and additive manufacturing techniques in fabricating a fracture-specific personalized orthopaedic cast. The hexagonal grid pattern of the cast played a key role in determining its mechanical properties enabling it to have an edge over its existing counterparts. Introduction of pores had reduced stress transfer and enhanced stress distribution properties while increasing its breathability and also minimizing the overall weight of cast. The mechanical stability of the designed cast was evaluated and approved using Finite Element Analysis. Furthermore, a Velcro-based lock and specific probe for adjuvant therapy such as photo/vibration therapy was integrated with the approved design and the cast was fabricated using fused deposition modelling technique.

Embodiments of the present invention will be described below in detail with reference to the accompanying drawings.

FIG. 1 illustrates a schematic representation 100 of the experimental strategy that includes the 3D scanning of fractured arm, ventilated designing of CAD modelling, and FEA simulation for mechanical robustness of the design and digital blueprint of the hand cast, according to an embodiment of the present disclosure.

3D Scanning of Fractured Arm:

The patient's arm was kept in an intact position and scanned using a 3D scanner (ARTEC EVA 3D). The scanned image was processed by isolating, refining, and enhancing the details of the region of interest using a software (ARTEC studio 13 Professional). This was exported in STL (Standard Triangular Language) format for complete personalization and CAD modelling. Processed scanned image of the patient's arm using Artec studio13 Professional software to enhance the details of region of interest (ROI) is shown in FIG. 2. FIG. 2 illustrates a processed scanned image 200 of the patient's arm with Region of Interest (ROI), according to an embodiment of the present disclosure.

CAD Modelling

Autodesk Meshmixer and Blender were used for CAD modelling. The former was used to transform the STL to create the outer surface with an offset of 0.5 cm to the hand. Porosity was introduced to the structure by generating a hexagonal grid pattern. This pattern was selected owing to the increased strength to weight ratio. Three designs were created-a control, a design that could mimic the conventional POP, two hexagonal grid patterned cast with edge thickness of 2.5 mm (P-2.5) and 3.5 mm (P-3.5) respectively as shown in FIG. 3. FIG. 3 illustrates CAD modelled cast designs 300, according to an embodiment of the present disclosure. In particular, FIG. 3A illustrates a CAD modelled cast design of a solid-non-porous cast (control), according to an embodiment of the present disclosure. FIG. 3B illustrates a CAD modelled cast design having P-2.5, according to an embodiment of the present disclosure. FIG. 3C illustrates a CAD modelled cast design having P-3.5, according to an embodiment of the present disclosure.

A Velcro-based lock was designed for the patterned cast using blender. Additionally, a slot for integrating ultrasound probe was created for different fracture-specific designs using 3D builder. All the designs were exported as STL format. These designs were then subjected to the FEA.

FEA Analysis

The patterned cast was analysed for any geometrical errors such as inexact edges, small faces, and tangency and then rectified using an ANSYS module, SpaceClaim. Further, ANSYS Workbench was used to create geometrical model and to generate finite meshes. This model was subjected to boundary conditions such as fixed support and point of action of force. The design with various material choices [i.e., Poly Lactic Acid (PLA), Acrylonitrile Butadiene Styrene (ABS), Nylon6, Glycol Modified Polyethylene Terephthalate (PETg)] was subjected to FEA. The cylindrical cast was fixed laterally to mimic the function of the lock and a range of compressive loads from 50 N, 100 N, 200 N and 300 N were applied from the top. A force of 300N was chosen to test the mechanical stability of the cast at extreme circumstance (i.e., when the patient falls again from a significant height in an upright position). These conditions were simulated, and the response was evaluated in terms of total deformation and equivalent stress. The response of the individual designs to the chosen materials was analysed and the best material and design were chosen.

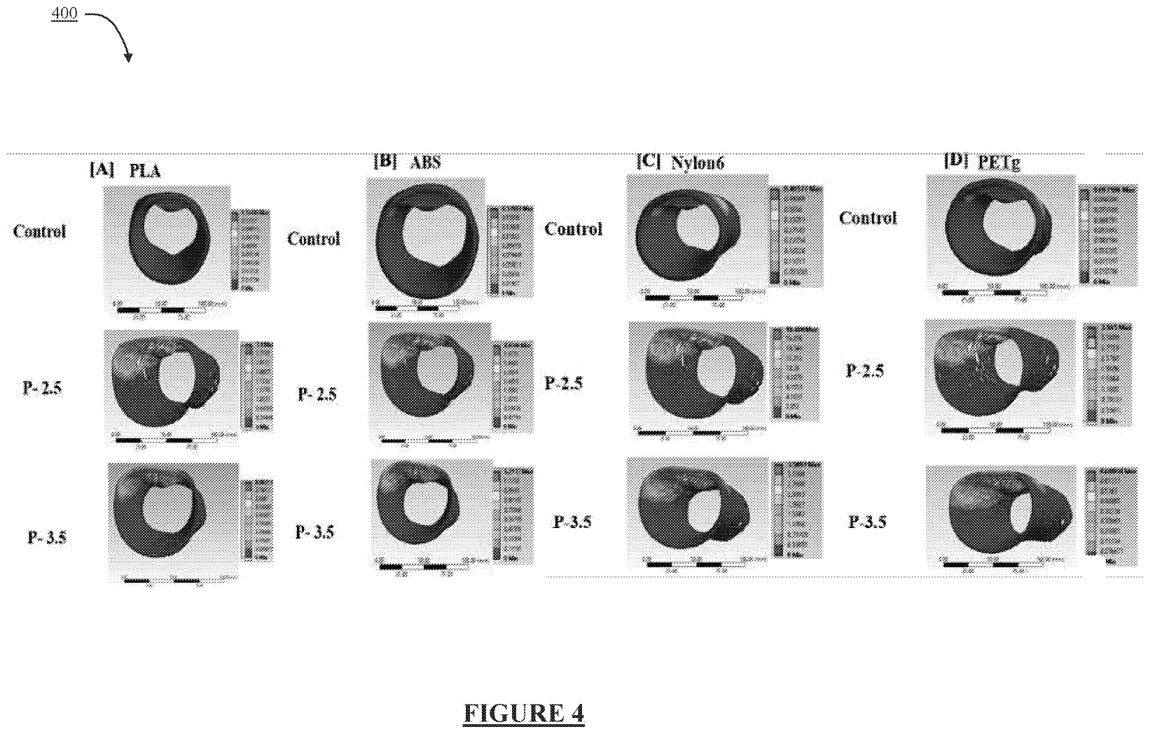

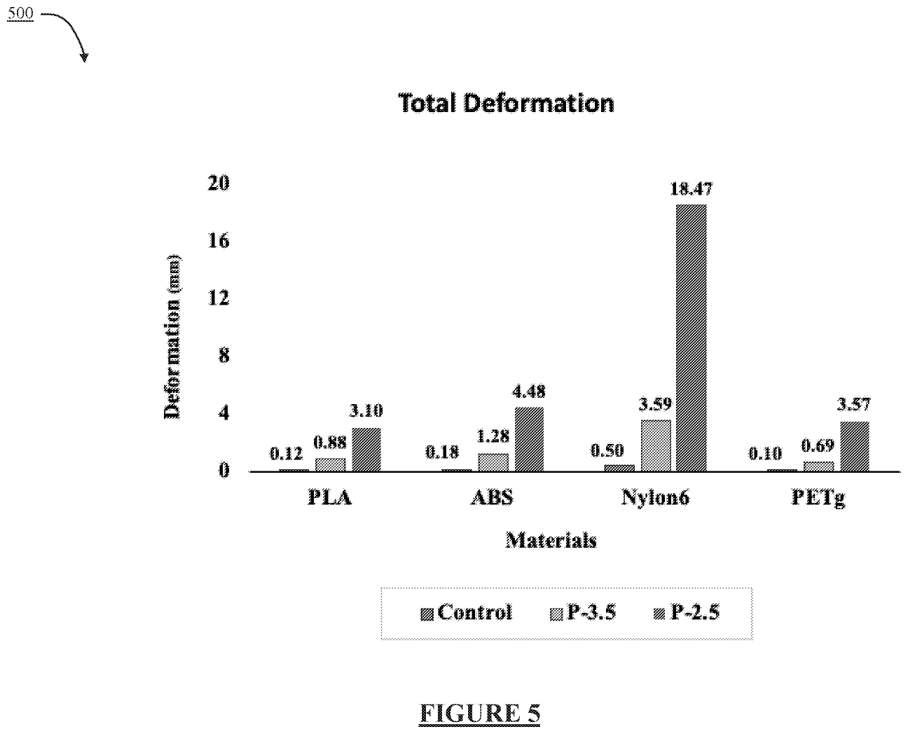

FIG. 4 illustrates Finite Element Analysis 400 of total deformation, according to an embodiment of the present disclosure. In particular, FIG. 4A illustrates an FEA analysis of total deformation in PLA, according to an embodiment of the present disclosure. FIG. 4B illustrates an FEA analysis of total deformation in ABS according to an embodiment of the present disclosure. FIG. 4C illustrates an FEA analysis of total deformation in Nylon6, according to an embodiment of the present disclosure. FIG. 4D illustrates an FEA analysis of total deformation in PETg, according to an embodiment of the present disclosure.

Total Deformation: The total deformation of the designed cast with and without patterns had been evaluated to determine the impact resistance of the cast under forward fall related injury with the maximum impact force of 300N. This demonstrates the ability of the cast to resist the deformation near or beyond 5 mm (offset distance) and thereby not transferring the stress to the underlying hand. The simulations were also carried out to check the deformation of the control design. Due to its complete solid structure, this design had the least deformation as expected. The design P-2.5 for the materials PLA, ABS, Nylon6 and PETg exhibited a total deformation of 3.1, 4.48, 18.47 and 3.57 mm respectively whereas the design P-3.5 deformed to 0.88, 1.28, 3.59 and 0.69 mm respectively corresponding to a load of 300 N (FIG. 4). The increased mechanical strength of the design P-3.5 may be due to the direct implication of increased solidity with the decreased pore size in the pattern compared to P-2.5. Maximum deformation of 18.47 mm of Nylon6 and 4.48 mm of ABS for the design P-2.5 exhibits higher risk of transferring the stress to the hand. In P-3.5, Nylon6 having a deformation of 3.59 mm also shows a low resistance to stress transfer. This might not only instigate physical discomfort to the patient, but also decelerate the fracture healing process. With respect to the design, P-3.5 outweighed P-2.5 pertaining to total deformation. FIG. 5 illustrates comparative analysis 500 of total deformation with design and material variability, according to an embodiment of the present disclosure. It was observed that both the material property and the design variability contributed almost equally to determining the extent of deformation of the cast.

Equivalent Stress Analysis

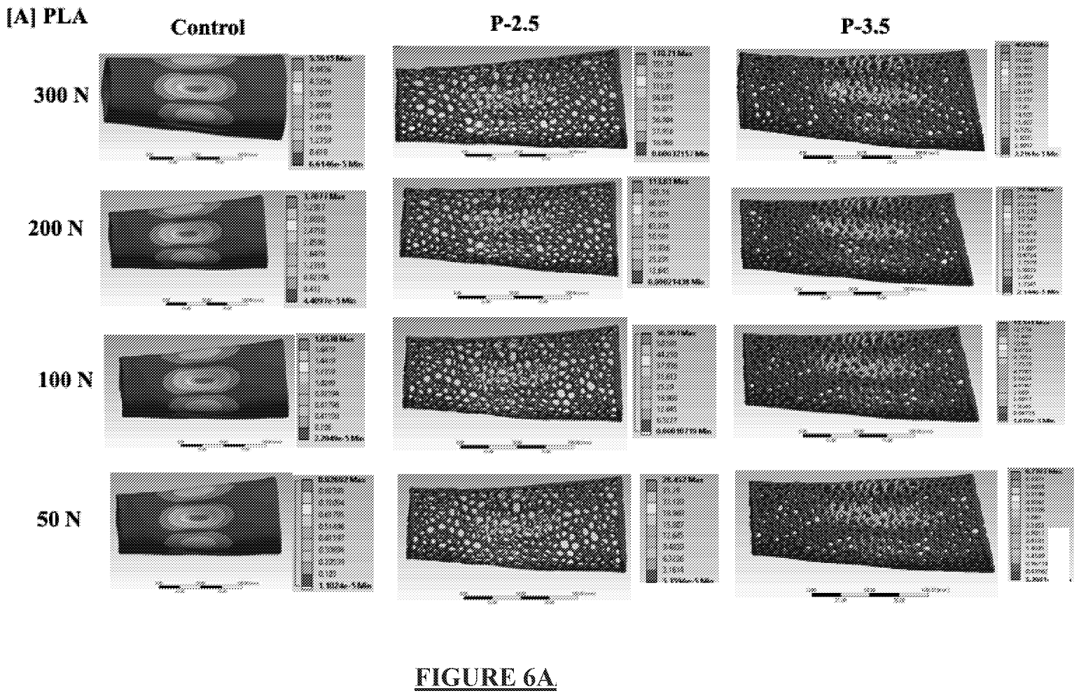

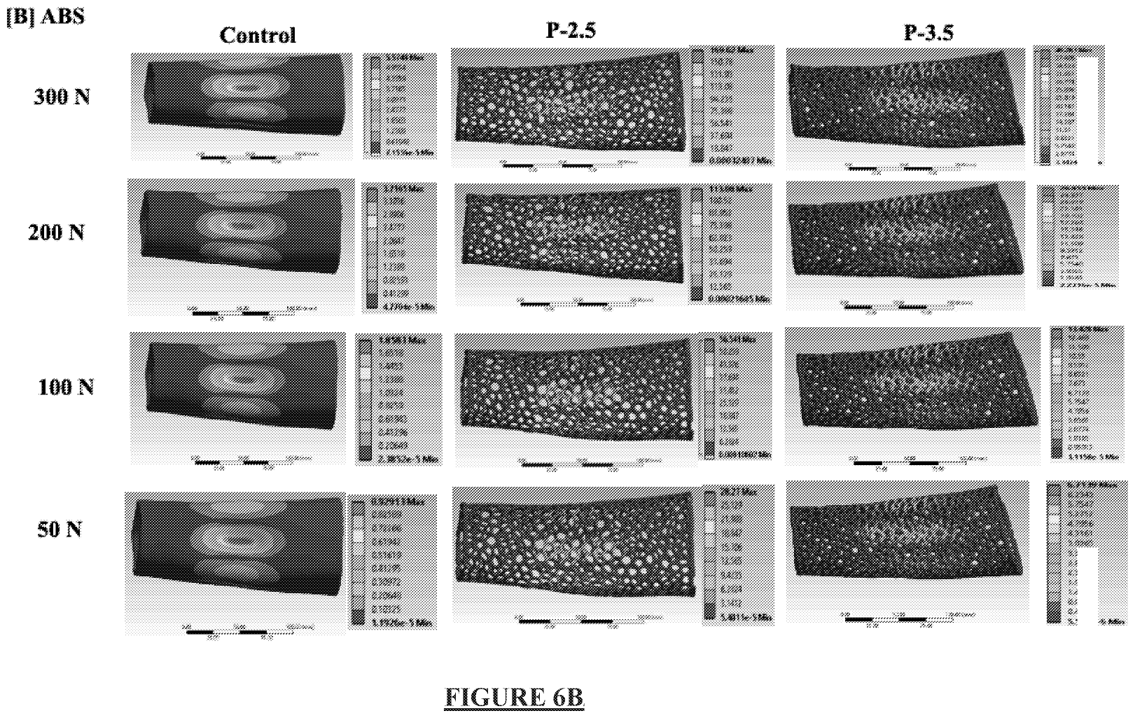

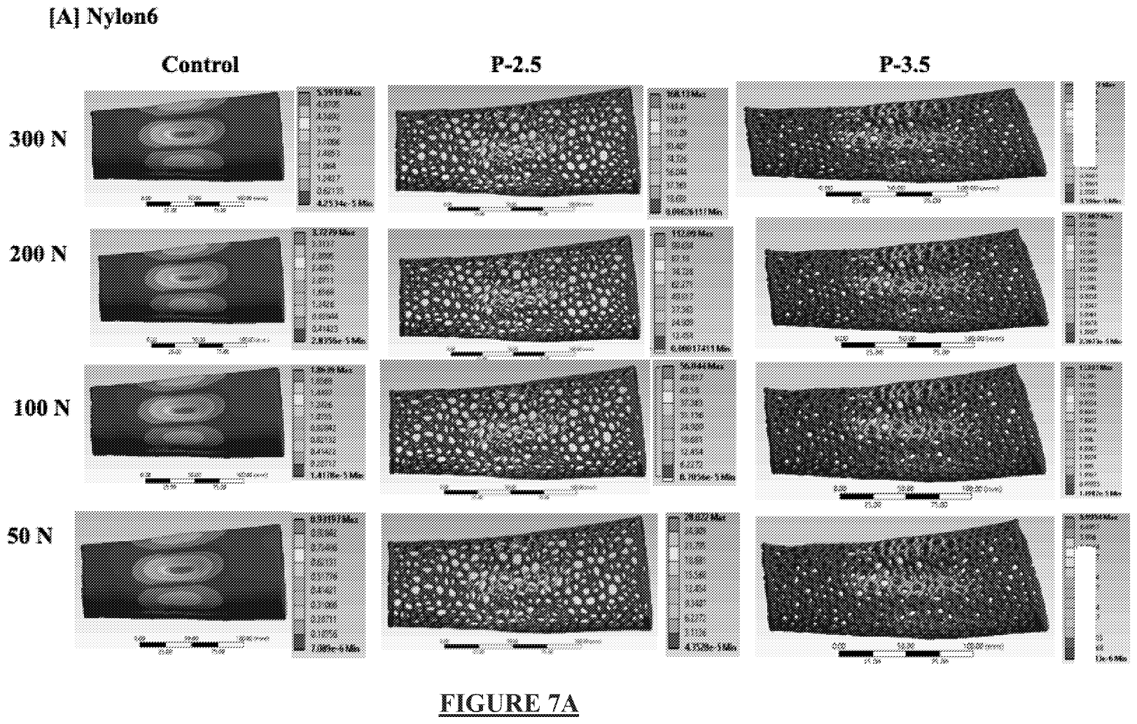

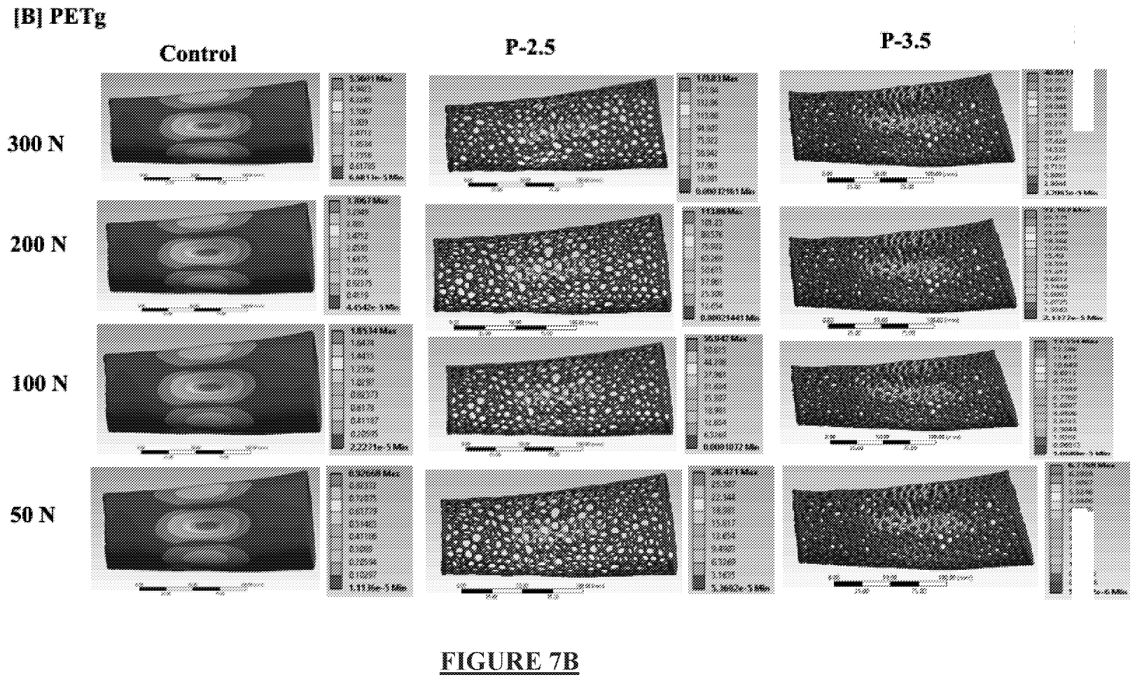

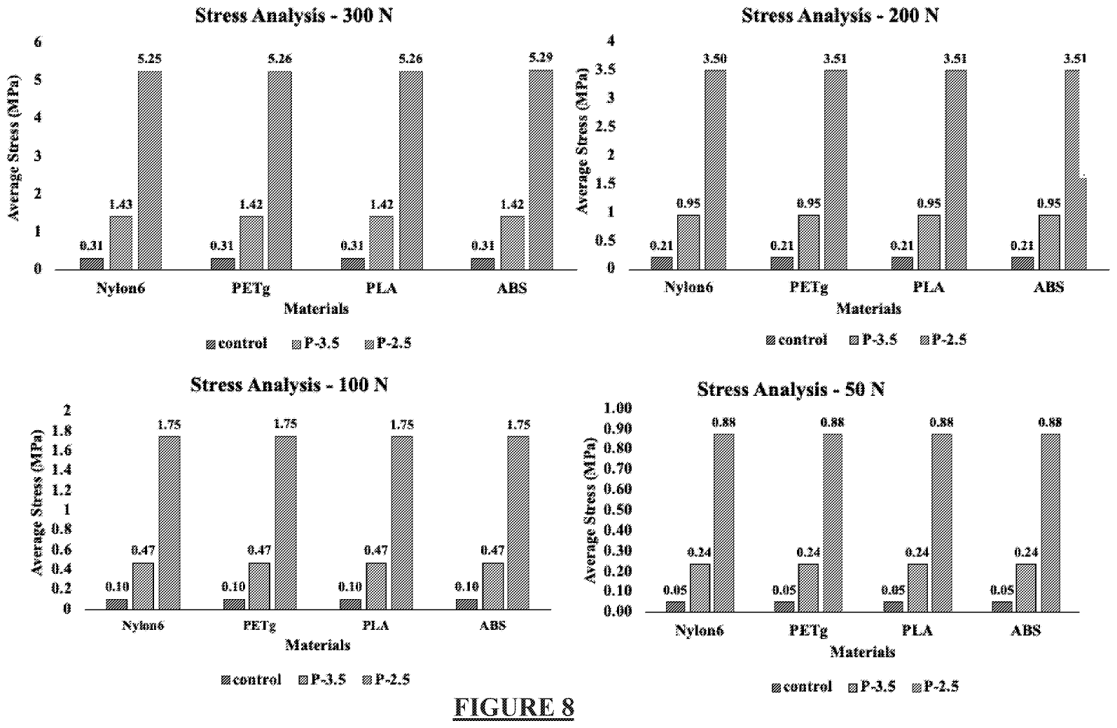

Equivalent stress analysis was performed to evaluate the stress distribution property of each design with respect to the material choice. The analysis was done for a range of forces with a minimum of 50 N to a maximum of 300 N. Though 300 N was considered as the impact force during forward fall, the purpose of including lower stress values was to measure the pattern of stress distribution for a multitude of loads. FIG. 6A, FIG. 6B, FIG. 7A, and FIG. 7B illustrate high concentration of forces for both the maximum and minimum load in the control design, proving its poor stress distribution capability. This structure being completely solid fails to transfer the stress across the design due to the absence of any interconnected pores. This might decrease the ability of the cast to withstand frequent application of load, thus leading to early breakage. Introduction of interconnected pores in the designs P-2.5 and P-3.5 contributed to their increased stress distribution property, thereby eliminating the concentration of load in a single region of the cast (FIG. 6). This improves the durability of the casts compared to that of the control design. It was also evident that P-3.5 had a slightly better stress distribution compared to that of P-2.5. This is because P-3.5 distributes its maximum stress to a greater number of intermediate values compared to that of P-2.5. Consequently, it has a reduced average stress value (1.42 MPa) than that of P-2.5 (5.26 MPa) for the highest load of 300 N (FIG. 8).

Thus, the pore size and the thickness of the line connecting the pores play a major role in distributing the stress across the cast, thereby improving the cast's mechanical performance. Further on, the analysis gave us an insight on the importance of the design. Although the material property displays a major role in determining the mechanical stability of a cast, the stress distributive property was nearly independent to the material of choice. This could be further proved with the fact that the variation in stress distribution was only seen across the designs and not within the same design for different materials (FIG. 8). This emphasizes that the stress distribution property of the cast is purely design dependent, highlighting the importance of different designs in fabricating an orthopaedic cast. Conclusively, from the comparative stress analysis (FIG. 6, FIG. 7, and FIG. 8), the design P-3.5 outperformed its counterpart and the influence of material choice was negligible in the case of stress distribution. Thus, this can be applied to various materials and designs and a database can be created for choosing an optimum design and a material for different fractures.

3D Printing

The best derived from the FEA was chosen for 3D printing using Fused Deposition Modelling (FDM) approach. The chosen biomaterial filament was loaded, and the design was printed using a commercial 3D printer (Ultimaker Extended) with a fill density of 50%.

Prototype Development

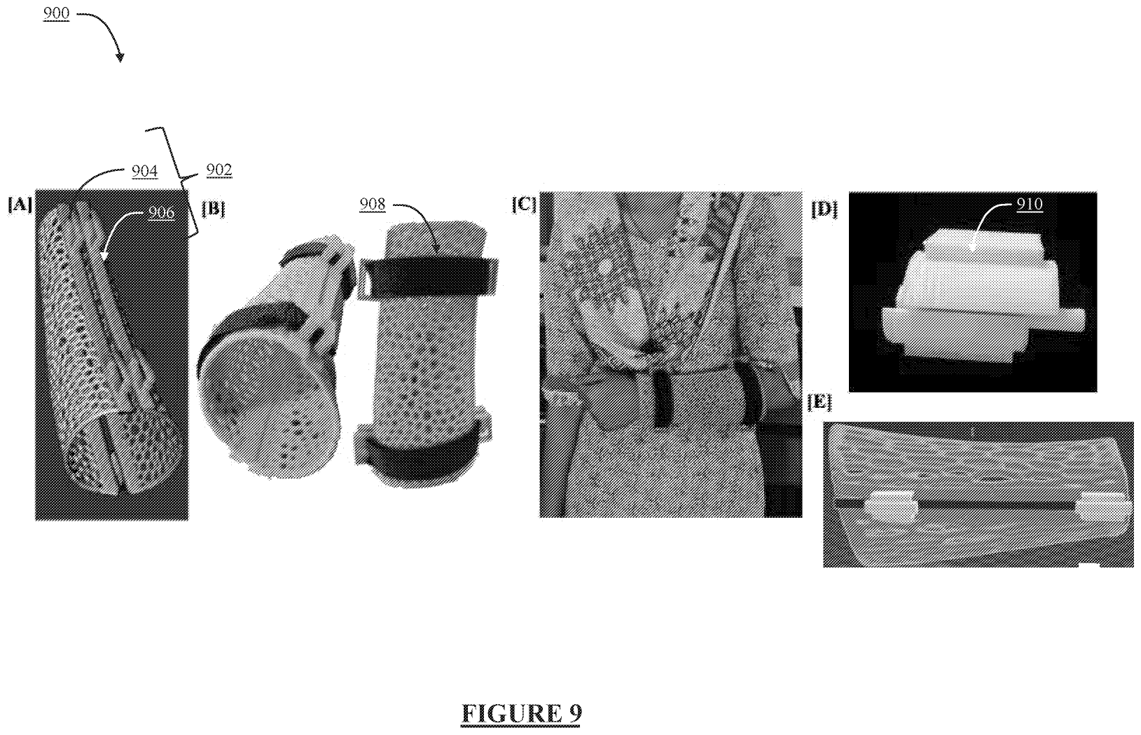

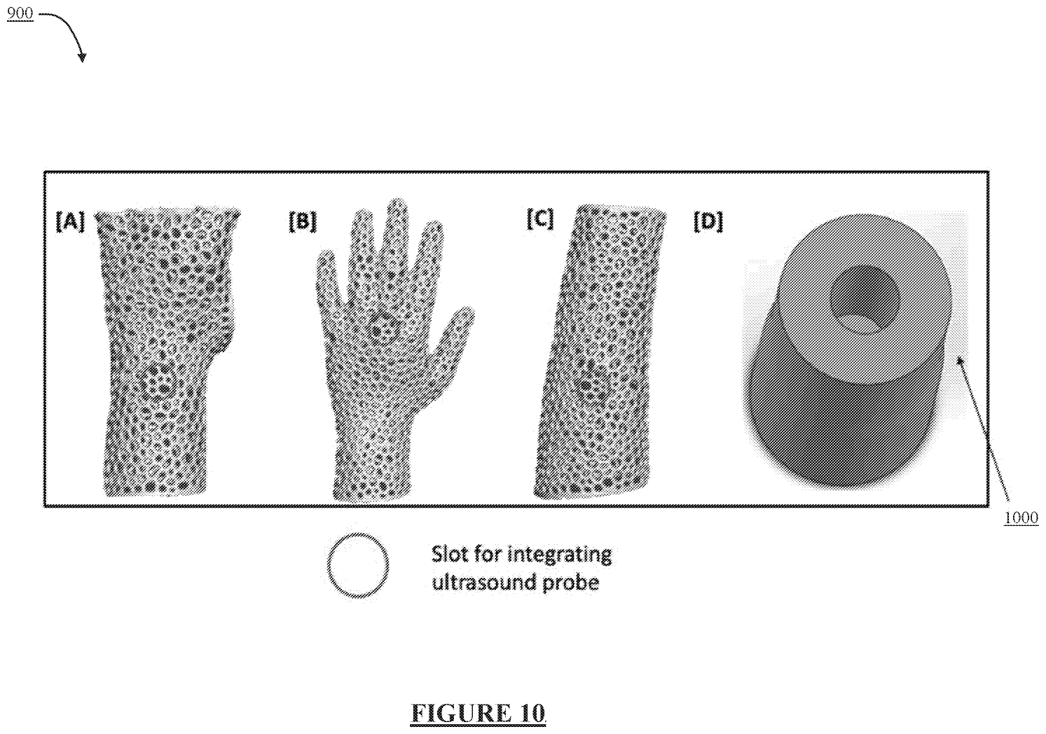

The design P-3.5 that was mechanically approved after FEA was chosen and the corresponding Velcro-based lock model was created using Blender. This model was 3D printed using Ultimaker-3 Extended commercial 3D printer with PLA filament. The parameters were optimized after several attempts to get a smooth finish with an aesthetic appeal. The printed cast weighed only about 118 g well below than its counterparts. The design that was mechanically approved after FEA, was imported and the Velcro-based lock was integrated after separating it into two equal halves. FIG. 9 illustrates an optimized mechanically robust design with Velcro based lock (A), a 3D-printed Cast (B), and Personalized hand-cast (C), according to an embodiment of the present disclosure.

As illustrated, the orthopaedic cast 900 may include a base component 902. The base component 902 may be formed of a first part 904 and a second part 906. The second part 906 may be adapted to be connected with the first part 904 such that the base component 902 is wrapped around the body part. The base component 902 may include a predefined grid pattern forming pores on a surface. In an embodiment, the base component 902 may include a hexagonal grid pattern. As would be appreciated by a person skilled in the art, in other embodiments, the base component 902 may include a grid pattern of any other profile than hexagon, without departing from the scope of the present disclosure.

The orthopaedic cast 900 may also include the Velcro-based lock 908 adapted to connect the first part 904 with the second part 906 to form the base component 902. In an embodiment, the 3D-printed orthopaedic cast 900 may be formed after generation of a Computer-Aided Design (CAD) and Finite Element Analysis (FEA) of the modelled cast.

In an embodiment, the orthopaedic cast 900 may be formed of at least one of PLA, ABS, Nylon6, and PETg. As would be appreciated by a person skilled in the art, in other embodiments, the orthopaedic cast 900 may be formed of any other polymeric or composite material, without departing from the scope of the present disclosure. In an embodiment, the orthopaedic cast 900 is water-resistant and sweat-resistant.

In an embodiment, the orthopaedic cast 900 may include at least one removable spacer 910 adapted to accommodate swelling of the body part. The removable spacer may be understood as a part that can be removed in case the body part is swollen. The removal of the spacer 910 would accommodate the swollen body part.

In an embodiment, the orthopaedic cast 900 may include at least one probe adapter adapted to deliver a localized adjuvant therapy to the body part. The localized adjuvant therapy may include, but is not limited to, photo therapy and vibration therapy.

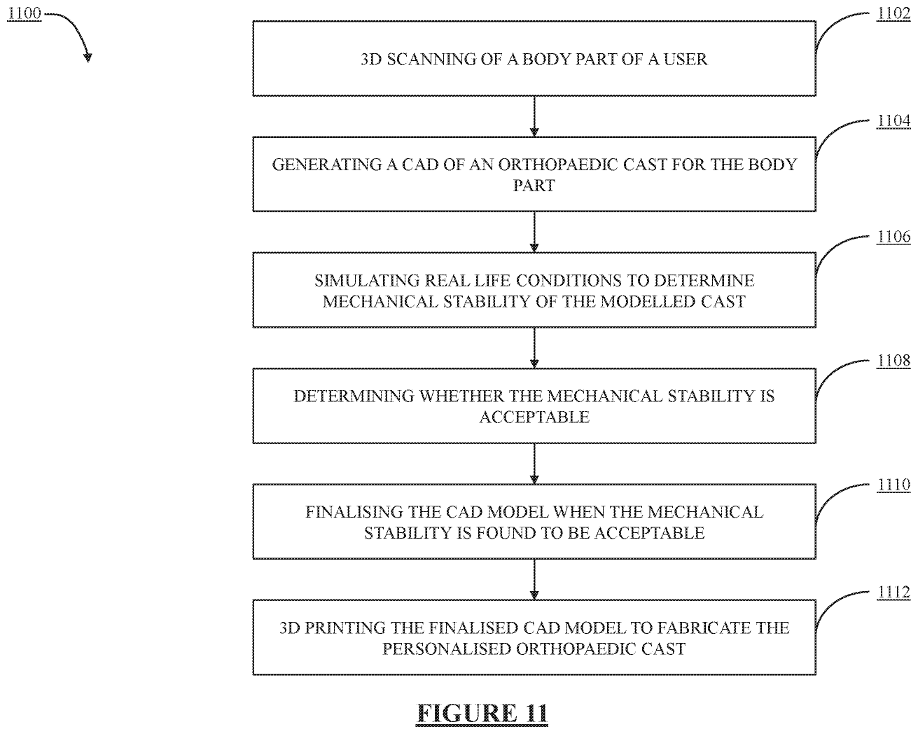

FIG. 10 illustrates fracture-specific breathable and washable orthopaedic casts 900 integrated with the probe adapter 1000 for adjuvant photo/vibration therapy of (A) Distal radius (wrist) fracture; (B) Metacarpal fracture; (C) Proximal radius fracture, according to an embodiment of the present disclosure. The casts 900 may include a slot to receive the probe adapter 1000. The probe adapter 1000 may be connected to a probe at one end. At the other end, the probe adapter 1000 may be inserted through the slot to be in contact with the body part for therapy.

FIG. 11 illustrates a flow chart depicting a method 1100 of fabricating the personalized orthopaedic cast 900, according to an embodiment of the present disclosure. For the sake of brevity, constructional and operational features of the orthopaedic cast 900 that are already explained in the description of FIG. 1 to FIG. 10 are not explained in detail in the description of FIG. 11.

At a block 1102, the method 1100 includes 3D scanning of a body part of a user. At a block 1104, the method 1100 includes generating the CAD of the orthopaedic cast 900 for the scanned body part. In an embodiment, the method 1100 may include identifying a Region of Interest (ROI) of the body part while scanning. Further, the method 1100 may include isolating, refining, and enhancing details of the ROI for generating the CAD of the orthopaedic cast 900.

At a block 1106, the method 1100 includes simulating real-life conditions to determine mechanical stability of the modelled cast. The mechanical stability may be determined through Finite Element Analysis (FEA). In an embodiment, the method 1100 may include determining impact resistance of the modelled cast by evaluating total deformation of the modelled cast. The method 1100 may further includes performing equivalent stress analysis of the modelled cast.

In an embodiment, the method 1100 may include simulating real-life conditions to analyze the modelled cast for a constructional error. The constructional error may include, but is not limited to, inexact edges, small faces, and tangency.

At a block 1108, the method 1100 includes determining whether the mechanical stability of the modelled cast is acceptable. At a block 1110, the method 1100 includes finalizing the CAD model when the mechanical stability of the modelled cast is found to be acceptable. In an alternate embodiment, the method 1100 may include determining that the mechanical stability of the modelled cast is not acceptable. In such an embodiment, the method 1100 may then include refining the modelled cast till the mechanical stability is found to be acceptable.

At a block 1112, the method 1100 includes 3D printing the finalised CAD model to fabricate the personalized orthopaedic cast 900. In an embodiment, the finalized CAD model may be 3D-printed by using Fused Deposition Modelling (FDM). In an embodiment, the finalized CAD model may be 3D printed with a fill density of about 50 percent for fabrication of the orthopaedic cast 900. In an embodiment, the finalised CAD model is 3D printed by using an additive manufacturing technique.

### Comparative Benefits and Advantages

Our approach yielded a 3D printed synthetic cast of any polymers that could supersede the traditional casts with its improved ideal properties thereby enhancing individual's physical comfort and provide a conducive environment for better healing of the fractured hand without compromising the daily activities such as bathing. The significant features of the cast are as follows;

1. Personalization: 3D scanning of fractured arm may provide complete personalization with best fit for immobilization and can be altered with respect to the area of fracture.
2. Designing of patterns with the FEA can be available in a pattern and fracture specific database.
3. Fracture specificity: Patterns with respect to fracture would enhance the stress distribution;
thereby avoid the localized stress on fractured bone.
4. Enhanced mechanical stability with uniform stress distribution. The mechanical stability would protect the immobilized bone tissue from the stress by distributing the localized force across the body.
5. Breathability avoids itching, sweating, rashes and secondary infections. Also, the breathability enables the physician/surgeon to monitor the healing process of any cutaneous wounds formed either by casting or by trauma-induced fracture
6. Light weight in comparison to existing casts. In an example, the weight of the orthopaedic cast is 118 g.
7. Water-resistant and sweat-resistant characteristics of the 3D printed cast enable the patients to perform the daily activities and prevent the itching, respectively.
8. Ease of casting through lock and key. The ease of designed lock and key in the orthopaedic cast 900 would prevent labor intensive casting procedure.
9. Removable spacers help to accommodate swelling immediately followed by injury/trauma and ease of spacers' removal from the cast during the healing process.
10. Space for probes in the 3D printed cast delivers localized adjuvant photo/vibration therapy simultaneously thereby enhance the healing process.

Hence, 3D implants have been developed as a personalized water-resistant, porous, rigid orthopaedic cast using 3D printing technique to overcome the current clinical cast-associated limitations. 3D printing technique offers the personalization of patient-specific perforated rigid casts and excludes multistep casting procedure along with the variability of hand molding.

While specific language has been used to describe the present disclosure, any limitations arising on account thereto, are not intended. As would be apparent to a person in the art, various working modifications may be made to the method in order to implement the inventive concept as taught herein. The drawings and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment.

We claim:

1. A method (1100) of fabricating a personalised orthopaedic cast (900), the method comprising:
3D scanning of a body part of a user;
generating a Computer Aided Design (CAD) of an orthopaedic cast (900) for the scanned body part;
simulating real-life conditions to determine mechanical stability of the modelled cast, wherein the mechanical stability is determined through Finite Element Analysis (FEA);
determining whether the mechanical stability of the modelled cast is acceptable;
finalising the CAD model when the mechanical stability of the modelled cast is found to be acceptable; and
3D printing the finalised CAD model to fabricate the personalised orthopaedic cast (900).

2. The method (1100) as claimed in claim 1, comprising:
identifying a Region of Interest (ROI) of the body part while scanning; and
isolating, refining, and enhancing details of the ROI for generating the CAD of the orthopaedic cast (900).

3. The method (1100) as claimed in claim 1, wherein determining the mechanical stability comprising:
determining impact resistance of the modelled cast by evaluating total deformation of the modelled cast; and
performing equivalent stress analysis of the modelled cast.

4. The method (1100) as claimed in claim 1, comprising simulating real-life conditions to analyse the modelled cast for a constructional error, wherein the constructional error comprising at least one of inexact edges, small faces, and tangency.

5. The method (1100) as claimed in claim 1, comprising:
determining that the mechanical stability of the modelled cast is not acceptable; and
refining the modelled cast till the mechanical stability is found to be acceptable.

6. The method (1100) as claimed in claim 1, wherein the finalised CAD model is 3D-printed by using Fused Deposition Modelling.

7. The method (1100) as claimed in claim 1, wherein the finalised CAD model is 3D printed with a fill density of about 50 percent for fabrication of the orthopaedic cast (900).

8. The method (1100) as claimed in claim 1, wherein the finalised CAD model is 3D printed by using an additive manufacturing technique.

9. A 3D-printed orthopaedic cast (900) for a body part of a user, the orthopaedic cast (900) comprising:
a base component (902) formed of a first part (904) and a second part (906) adapted to be connected with the first part (904) such that the base component (902) is wrapped around the body part, the base component (902) comprising a predefined grid pattern forming pores on a surface; and
a Velcro-based lock (908) adapted to connect the first part (904) with the second part (906) to form the base component (902),
wherein the 3D-printed orthopaedic cast (900) is formed after generation of a Computer-Aided Design (CAD) and Finite Element Analysis (FEA) of the modelled cast.

10. The orthopaedic cast (900) as claimed in claim 9, wherein the orthopaedic cast (900) is formed of a polymeric or composite material, wherein the polymeric material comprising at least one of Poly Lactic Acid (PLA), Acrylonitrile Butadiene Styrene (ABS), Nylon6, and Glycol Modified Polyethylene Terephthalate (PETg).

11. The orthopaedic cast (900) as claimed in claim 9, comprising at least one probe adapter (1000) adapted to deliver a localized adjuvant therapy to the body part, wherein the localized adjuvant therapy comprising at least one of photo therapy and vibration therapy.

12. The orthopaedic cast (900) as claimed in claim 9, comprising at least one removable spacer (910) adapted to accommodate swelling of the body part.

13. The orthopaedic cast (900) as claimed in claim 9, wherein the orthopaedic cast (900) is water-resistant and sweat-resistant.
